(19) United States
(12) Patent Application Publication
(10) Pub. No.: US 2015/0359715 A1
(43) Pub. Date: Dec. 17, 2015

(54) TRANS-RESVERATROL OR TRANS-RESVERATROL DERIVATIVE COMPOSITION

(71) Applicant: L'OREAL, Paris (FR)

(72) Inventors: Véronique CHEVALIER, Villecresnes (FR); Sandrine THEROUIN-KOELY, Clamart (FR)

(21) Appl. No.: 14/654,701

(22) PCT Filed: Dec. 20, 2013
(86) PCT No.: PCT/IB2013/061219

§ 371 Date: Jun. 22, 2015

Related U.S. Application Data
(60) Provisional application No. 61/751,052, filed on Jan. 10, 2013.

(30) Foreign Application Priority Data
Dec. 21, 2012 (FR) 12 62604

(51) Int. Cl. A61K 8/34; A61Q 19/08; A61Q 19/02; A61K 31/05; A61K 8/46; A61K 47/24; A61K 8/35; A61K 8/37; A61K 8/49; A61K 47/20; A61K 8/58; A61K 47/08; A61K 47/14; A61K 47/22; A61Q 17/04

(52) U.S. Cl. CPC A61K 8/347; A61Q 17/04; A61Q 19/08; A61Q 19/02; A61K 31/05; A61K 8/466; A61K 47/24; A61K 8/35; A61K 8/37; A61K 8/4966; A61K 47/20; A61K 8/585; A61K 47/08; A61K 47/14; A61K 47/22

(57) ABSTRACT

The invention relates to a cosmetic or dermatological composition which includes, in a physiologically acceptable medium, at least trans-resveratrol and/or at least one trans-resveratrol derivative, and an organic filtering system including at least one UVA filter and at least one UVB filter and/or at least one mixed UVA-UVB filter, said composition having an SPF no lower than 15 and a PPD no lower than 5. The invention also relates to a use of a filtering system which includes at least one UVA filter and at least one UVB filter and/or at least one mixed UVA-UVB filter for stabilising, in particular photostabilising, a composition including trans-resveratrol and/or at least one trans-resveratrol derivative.

The present invention relates to the field of compositions comprising trans-resveratrol and/or at least one trans-resveratrol derivative, in particular as an antioxidant, anti-aging agent or depigmenting agent.

Resveratrol is a polyphenol of the stilbene class which exists in a cis form and a trans form represented below.

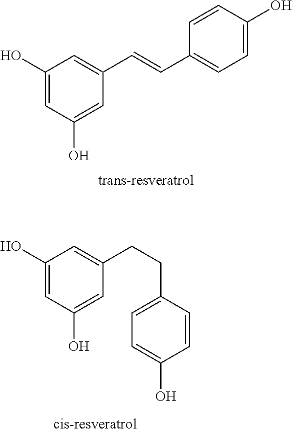 [SMILES: Oc1ccc(/C=C/c2cc(O)cc(O)c2)cc1 ; Oc1ccc(CCc2cc(O)cc(O)c2)cc1]

Trans-resveratrol is known to have numerous biological properties, such as an antioxidant, anti-inflammatory or antitumor activity.

However, trans-resveratrol is a photosensitive molecule which is irreversibly converted into its cis isomer under exposure to light, however the trans isomer is the most active form.

Thus, there remains a need to have a composition containing trans-resveratrol, which is stable in particular with respect to light.

The inventors have noted that it is possible to obtain such a composition by introducing UVA- and UVB-screening agents into said composition.

Thus, a subject of the present invention, according to a first of its aspects, is a cosmetic or dermatological composition comprising, in a physiologically acceptable medium, at least trans-resveratrol and/or at least one trans-resveratrol derivative, and an organic screening system comprising at least one UVA-screening agent and at least one UVB-screening agent and/or at least one mixed UVA-UVB-screening agent, said composition having an SPF greater than or equal to 15 and a PPD greater than or equal to 5.

Preferably, the trans-resveratrol or trans-resveratrol derivative is present in a content ranging from 0.001% to 10% by weight relative to the total weight of the composition.

In particular, the composition according to the invention is characterized in that the SPF/PPD ratio ranges from 1 to 3, preferably from 1.3 to 2.7 and better still from 1.5 to 2.5.

According to a second aspect, the invention is directed toward a process for stabilizing, in particular photostabilizing, a composition comprising trans-resveratrol and/or at least one trans-resveratrol derivative, comprising a step during which an organic screening system comprising at least one UVA-screening agent and at least one UVB-screening agent and/or at least one mixed UVA-UVB-screening agent is added to said composition in an amount such that the SPF of the composition is brought to a value greater than or equal to 15 and the PPD of the composition is brought to a value greater than or equal to 5.

The invention is also directed toward the use of a screening system comprising at least one UVA-screening agent and at least one UVB-screening agent and/or at least one mixed UVA-UVB-screening agent, for stabilizing, in particular photostabilizing, a composition comprising trans-resveratrol and/or at least one trans-resveratrol derivative.

This use comprises in particular the addition, to said composition, of said organic screening system in an amount such that the SPF of the composition is brought to a value greater than or equal to 15 and the PPD of the composition is brought to a value greater than or equal to 5.

Surprisingly, the inventors have noted that the trans-resveratrol present in the composition according to the invention is particularly stable after having been exposed to light.

For the purposes of the present invention, the term “trans-resveratrol derivative” is intended to mean the esters, glucosides and phosphates of trans-resveratrol.

These compounds all have the same biological properties as trans-resveratrol and have trans stereochemistry. Just like trans-resveratrol, they are photosensitive and are irreversibly converted to their cis isomer under exposure to light.

In the remainder of the description, the term “trans-resveratrol” is intended to mean trans-resveratrol or at least one trans-resveratrol derivative or a mixture of trans-resveratrol and trans-resveratrol derivative(s).

For the purposes of the present invention, the term “stable” is intended to mean a composition in which the trans-resveratrol undergoes little degradation particularly under exposure to light, in particular by conversion into its cis isomer. Preferably, a stable composition according to the invention comprises at least 70% by weight of resveratrol, advantageously at least 75% by weight of resveratrol, in particular at least 80% by weight of resveratrol, relative to the total weight of resveratrol initially introduced into the composition.

For the purposes of the invention, the term “SPF” is intended to mean the sun protection factor, which measures the level of protection against UVB radiation. The value of the SPF corresponds to the ratio between the minimum time necessary to obtain sunburn with a sunscreen composition and the minimum time without product.

It is expressed mathematically by the ratio of the UV radiation dose necessary to reach the erythemogenic threshold with the UV-screening agent to the UV radiation dose necessary to reach the erythemogenic threshold without UV-screening agent. This factor thus concerns the efficacy of the protection, the biological spectrum of action of which is centered in the UVB range, and consequently accounts for the protection with respect to this UVB radiation.

To characterize the protection with respect to UVA, the PPD (persistent pigment darkening) method, which measures the skin color observed 2 to 4 hours after exposure of the skin to UVA radiation, is particularly recommended and used. This method has been adopted since 1996 by the Japanese Cosmetic Industry Association (JCIA) as official test procedure for the UVA labeling of products and is frequently used by test laboratories in Europe and the United States; (Japan Cosmetic Industry Association Technical Bulletin. Measurement Standards for UVA protection efficacy. Issued Nov. 21, 1995 and effective as of Jan. 1, 1996).

The UVA PPD sun protection factor (UVAppd PF) is expressed mathematically by the ratio of the dose of UVA radiation necessary to reach the pigmentation threshold with the UV-screening agent (MPPDp) to the dose of UVA radiation necessary to reach the pigmentation threshold without UV-screening agent (MPPDnp).

$${UVA}_{PPD} PF = \begin{matrix}MPPDp \\ MPPDnp\end{matrix}$$

The present application also relates, according to another of its aspects, to the cosmetic use of an effective amount of trans-resveratrol or of at least one trans-resveratrol derivative as previously defined, for combating the signs of skin aging, in particular induced by an oxidative stress.

Indeed, oxidative stress is in particular linked to external agents such as ultraviolet radiation, pollution, thermal stress or else various toxins such as chemical agents with which the skin may be in contact (tobacco, heavy metals, atmospheric pollutants, etc.).

In particular, the use according to the invention aims to reduce the signs of skin aging, in particular the signs of skin aging of actinic origin, such as photoaging.

Among the signs of skin aging, in particular induced by an oxidative stress, in particular of actinic origin, mention is in particular made of a loss of firmness and/or elasticity and/or tone and/or suppleness of the skin, the formation of wrinkles and fine lines, expression lines, in particular on the forehead and in the space between the eyebrows, perioral wrinkles and fine lines, and/or slackening in the area around the lips, in particular in the top lip area (area between the top lip and the nose), a dull appearance of the complexion, and the wizened appearance of the skin.

This use will in particular be intended for individuals with a mature, or even very mature, skin.

According to the invention, the term “mature skin” is intended to mean in particular individuals who are at least 40 years old.

According to the invention, the term “very mature skin” is intended to mean in particular individuals who are at least 50 years old, in particular at least 60 years old, or even 65 years old.

Expression wrinkles are produced due to the effect of the strain exerted on the skin by the skin muscles that allow facial expressions. Depending on the shape of the face, the frequency of facial expressions and possible tics, they may appear from childhood. Certain environmental factors, such as oxidative stress, exposure to the sun, and also age, are not involved in creating them, but may make them deeper and make them permanent. Expression wrinkles are characterized by the presence of grooves around the orifices formed by the nose (nasal grooves), the mouth (perioral wrinkles and “sour-face” wrinkles) and the eyes (crow's-feet wrinkles), around which are the skin muscles, and also between the eyebrows (glabella wrinkles or lion wrinkles) and on the forehead. In particular, it will be sought to prevent and/or smooth out the wrinkles on the forehead and the area between the eyebrows.

The wizened appearance of the skin is characterized by a change in the visual appearance, and also in the behavior of the skin to the touch. More specifically, the skin visually takes on the appearance of cigarette paper, giving it an appearance similar to that of a sheet of papyrus. In addition, when it is pinched gently between the thumb and index finger, the skin forms numerous fine sharp folds having the appearance of crumpled paper. Finally, the feel of the skin shows that its surface parts appear to be floating on the deep parts, giving the skin, in the very advanced stage of wizened appearance, the appearance of worn-looking paper. The wizened appearance of the skin is visible on the face and even more characteristic on the back of the hands of the elderly.

The compositions in accordance with the invention that are intended for preventing and/or smoothing out expression wrinkles will be applied to the face.

The compositions in accordance with the invention that are intended for preventing and/or treating the wizened appearance of the skin will in particular be applied to the back of the hands.

The compositions in accordance with the invention that are intended for decreasing the appearance and/or the visibility of the pores will be applied in particular to the T zone (forehead, nose, cheeks, chin)

The use of antioxidant active agents can prove to be useful in anti-aging compositions or else in depigmenting compositions.

The use according to the invention can also be more particularly intended for individuals with oily skin.

The compositions in accordance with the invention may be makeup or care products for keratin materials, in particular the skin.

More specifically, the makeup products may be of the type such as foundations, face powders, eyeshadows, concealer products or blushers, or alternatively a body makeup product or a skin coloring product.

The skincare products may be a protective, treating or care composition for the face, for the hands, for the feet, for the major anatomical folds or for the body, for example day creams, night cream, antisun composition, protective or care body milks, after-sun milks, skincare lotion, gel or foam, or artificial tanning composition; or an aftershave composition.

The present invention also relates to a process for the cosmetic treatment of the skin and/or skin appendages, intended for preventing and/or treating skin aging, in particular induced by an oxidative stress, comprising at least one step consisting in applying, to skin and/or skin appendages exhibiting signs of skin aging induced by an oxidative stress, at least an effective amount of at least one composition according to the invention.

The present invention also relates to a process for the cosmetic treatment of the skin and/or skin appendages, aimed at protecting the skin and/or skin appendages against the effects of oxidative stress, in particular against the effects of UV radiation and/or toxins and/or pollution, comprising at least one step consisting in applying, to the skin and/or skin appendages, at least an effective amount of at least one composition according to the invention. The composition according to the invention is suitable for topical application.

The term “skin” is intended to mean the skin of the face and/or of the body, the scalp and the semi-mucous membranes (lips). Preferably, it involves the skin of the face and/of the body and/or the lips.

The term “skin appendages” is intended to mean head hair, body hair, eyelashes, nails and preferably head hair.

Finally, the present invention relates to a cosmetic treatment process intended for preventing skin irritation induced by an oxidative stress, comprising at least one step consisting in applying, to skin which may be subjected to an oxidative stress, at least an effective amount of at least one composition as previously defined.

The term “oxidative stress” as used in the present application covers all of the damage caused by free radicals in a subject.

The extent of the damage generated by this oxidative stress depends on the rapidity with which the free radicals are created and subsequently inactivated by antioxidants.

Trans-Resveratrol

Resveratrol exists in the natural state in its cis and trans form, but also in other forms, for instance a glucosylated form. It is present in many plants and fruits. It is found in particular in Japanese knotweed (Fallopia japonica also known as Polygonum cupistadum or else Reynoutria japonica) or in grapes, for instance those from the grapevine species Vitis vinifera. More particularly, resveratrol is found, for example, in blackberries, wine or peanuts.

Generally, resveratrol is found in the following plant families. Vitaceae, Myrtaceae, Dipterocarpaceae, Cyperaceae, Gnetaceae, Fabaceae, Pinaceae, Polygonaceae, Moraceae, Fagaceae, Liliaceae, etc.

The trans-resveratrol present in the composition according to the invention may be in the form of a plant extract containing trans-resveratrol.

According to one particular embodiment of the invention, said trans-resveratrol and/or at least one trans-resveratrol derivative is present in the composition according to the invention in a content ranging from 0.01% to 5% by weight, preferably from 0.05% to 2% by weight, relative to the total weight of the composition.

For the purposes of the invention, the term “content” is intended to mean active material content.

The present invention is also directed toward a process for preparing said composition.

Organic Screening Agents

As previously specified, a composition according to the invention comprises an organic screening system comprising at least one UVA-screening agent and at least one UVB-screening agent and/or at least one mixed UVA-UVB-screening agent.

UVA-Screening Agents

The UVA-screening agent(s) according to the present invention can be chosen from the following screening agents.

1) Hydrophobic UVA-Screening Agents

Dibenzoylmethane Derivatives:
Butylmethoxydibenzoylmethane sold in particular under the trade name Parsol 1789 by DSM Nutritional Products, Inc.;
Isopropyl dibenzoylmethane;

Aminobenzophenones:
n-Hexyl 2-(4-diethylamino-2-hydroxybenzoyl)benzoate sold in particular under the trade name Uvinul A+ by BASF;

Anthranilic Derivatives:
Menthyl anthranilate sold in particular under the trade name Neo Heliopan MA by Symrise;

4,4-Diarylbutadiene Derivatives:
1,1-Dicarboxy-(2,2′-dimethylpropyl)-4,4-diphenylbutadiene;

Merocyanine Derivatives:
Octyl 5-N,N-diethylamino-2-phenylsulfonyl-2,4-pentadienoate.

2) Water-Soluble UVA-Screening Agents
Camphor derivatives such as terephthalylidenedicamphorsulfonic acid produced under the name Mexoryl SX by Chimex;
Bis-benzoazolyl derivatives as described in patents EP 669 323, and U.S. Pat. No. 2,463,264 and more particularly the compound disodium phenyldibenzimidazotetrasulfonate sold under the trade name Neo Heliopan AP by Symrise.

According to one preferred embodiment, said at least one UVA-screening agent is chosen from hydrophobic dibenzoylmethane derivatives, water-soluble camphor derivatives, and mixtures thereof.

More particularly, said at least one UVA-screening agent is chosen from butyl methoxydibenzoylmethane, terephthalylidenedicamphorsulfonic acid, and mixtures thereof. Preferably, a composition according to the invention is characterized in that said at least one UVA-screening agent is present in a content ranging from 1% to 20%, in particular from 2% to 15% by weight, relative to the total weight of the composition.

According to another embodiment, said at least one UVA-screening agent is a water-soluble UVA-screening agent.

UVB-Screening Agents

The UVB-screening agent(s) according to the present invention can be chosen from the following screening agents.

1) Hydrophobic UVB-Screening Agents

Para-Aminobenzoates:
Ethyl PABA;
Ethyl dihydroxypropyl PABA;
Ethylhexyl dimethyl PABA (Escalol 507 from ISP).

Salicylic Derivatives:
Homosalate sold in particular under the name Eusolex HMS by Rona/EM Industries;
Ethylhexyl salicylate sold in particular under the name Neo Heliopan OS by Symrise;
Dipropylene glycol salicylate sold in particular under the name Dipsal by Scher;
TEA salicylate and under the name Neo Heliopan TS by Symrise.

Cinnamates
Ethylhexyl methoxycinnamate sold in particular under the trade name Parsol MCX by DSM Nutritional Products, Inc.;
isopropyl methoxycinnamate;
Isoamyl methoxycinnamate sold in particular under the trade name Neo Heliopan E 1000 by Symrise;
Diisopropyl methylcinnamate;
Cinnoxate;
Glyceryl ethylhexanoate dimethoxycinnamate

β,β′-Diphenylacrylate Derivatives:
Octocrylene, sold in particular under the trade name Uvinul N539 by BASF;
Etocrylene sold in particular under the trade name Uvinul N35 by BASF.

Benzylidenecamphor Derivatives:
3-Benzylidenecamphor produced under the name Mexoryl SD by Chimex;
Methylbenzylidenecamphor sold in particular under the name Eusolex 6300 by Merck;
Polyacrylamidomethylbenzylidenecamphor produced under the name Mexoryl SW by Chimex.

Triazine Derivatives:
Ethylhexyltriazone sold in particular under the trade name Uvinul T150 by BASF;
Diethyihexylbutamidotriazone sold in particular under the trade name Uvasorb HEB by Sigma 3V;
2,4,6-tris(dineopentyl 4′-aminobenzalmalonate)-s-triazine;
2,4,6-tris(diisobutyl 4′-aminobenzalmalonate)-s-triazine;
2,4-bis(dineopentyl 4′-aminobenzalmalonate)-6-(n-butyl 4′-aminobenzoate)-s-triazine;
2,4-bis(n-butyl 4′-aminobenzoate)-6-(aminopropyltrisiloxane)-s-triazine;
   the symmetrical triazine screening agents described in the patent U.S. Pat. No. 6,225,467, application WO 2004/085412 (see compounds 6 and 9) or the document “Symetrical Triazine Derivatives” IP.COM Journal, IP.COM Inc West Henrietta, N.Y., US (Sep. 20, 2004) in particular 2,4,6-tris-(diphenyl)-1,3,5-triazine (in particular 2,4,6-tris(diphenyl-4-yl-1,3,5-triazine) and 2,4,6-tris(terphenyl)-1,3,5-triazine, the latter two screening agents being described in the Beiersdorf applications WO 06/035000, WO 06/034982, WO 06/034991, WO 06/035007, WO 2006/034992, WO 2006/034985).

Imidazoline Derivatives:
Ethylhexyl dimethoxybenzylidene dioxoimidazoline propionate.

Benzalmalonate Derivatives:
Polyorganosiloxanes containing benzalmalonate functions, for instance Polysilicone-15, sold in particular under the trade name Parsol SLX by DSM Nutritional Products, Inc.;
Dineopentyl 4′-methoxybenzalmalonate.

2) Water-Soluble UVB-Screening Agents

The following p-aminobenzoic acid (PABA) derivatives:
PABA,
Glyceryl PABA, and
PEG-25 PABA sold in particular under the trade name Uvinul P25 by BASF,
Phenylbenzimidazole sulfonic acid sold in particular under the trade name Eusolex 232 by Merck,
ferulic acid,
salicylic acid,
DEA methoxycinnamate,
benzylidenecamphorsulfonic acid produced under the name Mexoryl SL by Chimex,
camphorbenzalkonium methosulfate produced under the name Mexoryl SO by Chimex.

According to one preferred embodiment, the at least one UVB-screening agent included in a composition according to the invention is a hydrophobic UVB-screening agent.

In particular, said at least one hydrophobic UVB-screening agent is chosen from salicylic derivatives, cinnamates, β,β′-diphenylacrylate derivatives, triazine derivatives, and mixtures thereof.

Preferably, the at least one UVB-screening agent is chosen from ethylhexyl salicylate, ethylhexyl methoxycinnamate, octocrylene, ethylhexyl triazone, and mixtures thereof.

Preferably, a composition according to the invention is characterized in that said at least one UVB-screening agent is present in a content ranging from 1% to 20%, in particular from 5% to 15%, and preferably from 7% to 10% by weight, relative to the total weight of the composition.

According to one particular variant of the invention, said organic screening system used in the invention contains or even consists of one or more mixed UVA-UVB-screening agent(s).

Advantageously, said organic screening system used in the invention contains one or more UVA-screening agents, one or more UVB-screening agents and one or more mixed UVA-UVB-screening agents.

Mixed UVA- and UVB-Screening Agents

Said at least one mixed UVA-UVB-screening agent can be chosen from the following screening agents.

1) Hydrophobic Mixed UVA- and UVB-Screening Agents

Benzophenone Derivatives
Benzophenone-1 sold in particular under the trade name Uvinul 400 by BASF;
Benzophenone-2 sold in particular under the trade name Uvinul D50 by BASF;
Benzophenone-3 or oxybenzone sold in particular under the trade name Uvinul M40 by BASF;
Benzophenone-6 sold in particular under the trade name Helisorb 11 by Norquay;
Benzophenone-8 sold in particular under the trade name Spectra-Sorb UV-24 by American Cyanamid;
Benzophenone-10;
Benzophenone-11;
Benzophenone-12.

Phenylbenzotriazole Derivatives:
Drometrizole trisiloxane sold in particular under the name Silatrizole by Rhodia Chimie or produced under the name Mexoryl XL by the company Chimex;
Methylenebis(benzotriazolyl)tetramethylbutylphenol sold in solid form in particular under the trade name Mixxim BB/100 by Fairmount Chemical, or in micronized form as an aqueous dispersion in particular under the trade name Tinosorb M by Ciba Specialty Chemicals.

Bis-Resorcinyl Triazine Derivatives:
Bis(ethylhexyloxyphenol)methoxyphenyltriazine sold in particular under the trade name Tinosorb S by Ciba-Geigy.

Benzoxazole Derivatives:
2,4-Bis [5-(1-dimethylpropyl)benzoxazol-2-yl-(4-phenyl)imino]-6-(2-ethylhexyl)imino-1,3,5-triazine, sold in particular under the name Uvasorb K2A by Sigma 3V.

2) Water-Soluble Mixed UVA- and UVB-Screening Agents

Benzophenone derivatives comprising at least one sulfonic radical, such as
Benzophenone-4 sold in particular under the trade name Uvinul MS 40 by BASF,
Benzophenone-5, and
Benzophenone-9.

Preferably, said at least one mixed UVA-UVB-screening agent according to the invention is a hydrophobic screening agent.

In particular, said mixed UVA-UVB-screening agent is chosen from phenylbenzotriazole derivatives and bis-resorcinyl triazine derivatives, it is preferably chosen from drometrizole trisiloxane, bis-ethylhexyloxyphenol methoxyphenyl triazine, and mixtures thereof.

Preferably, a composition according to the invention is characterized in that said at least one mixed UVA-UVB-screening agent is present in a content ranging from 1% to 20%, in particular from 1% to 10%, and preferably from 1% to 4% by weight, relative to the total weight of the composition.

According to one particular embodiment, a composition of the invention is characterized in that said organic screening system comprises at least one screening agent chosen from terephthalylidenedicamphorsulfonic acid, drometrizole trisiloxane, and mixtures thereof.

According to a first preferred variant, the composition according to the invention comprises octocrylene, ethylhexyltriazone (Uvinul T150), drometrizole trisiloxane (Mexoryl XL), butyl methoxydibenzoylmethane (Avobenzone), terephthalylidenedicamphorsulfonic acid (Mexoryl SX) with or without TiO₂

According to a second preferred variant, the composition according to the invention comprises drometrizole trisiloxane (Mexoryl XL), ethylhexyl methoxycinnamate, terephthalylidenedicamphorsulfonic acid (Mexoryl SX) and TiO₂ which has been treated, for example with aluminum stearate.

According to a third preferred variant, the composition according to the invention comprises drometrizole trisiloxane (Mexoryl XL), homosalate, ethylhexyltriazone (Uvinul T150), ethylhexyl salicylate, bis-ethylhexyloxyphenol methoxyphenyl triazine, butyl methoxydibenzoylmethane (Avobenzone), and terephthalylidenedicamphorsulfonic acid (Mexoryl SX).

Advantageously, a composition according to the invention, and more particularly when it is in the form of an emulsion, is characterized in that said organic screening system comprises at least one water-soluble screening agent and at least one hydrophobic screening agent, said water-soluble screening agent preferably being a UVA-screening agent.

Thus, the organic screening system preferably comprises at least one water-soluble UVA-screening agent and at least one hydrophobic UVB-screening agent; in particular, it consists of the combination of at least one water-soluble UVA-screening agent and at least one hydrophobic UVB-screening agent.

More particularly, the composition according to the invention comprises the following UV-screening agent mixtures by weight relative to the total weight of the composition:
from 1% to 5% of butyl methoxydibenzoylmethane, from 0.1% 3% of ethylhexyltriazone, from 0.5% to 4% of terephthalylidenedicamphorsulfonic acid, from 5% to 10% of octocrylene, from 0.5% to 4% of drometrizole trisiloxane and from 0.1% to 3% of TiO₂ relative to the total weight of the composition;
from 1% to 5% of butyl methoxydibenzoylmethane, from 3% to 7% of ethylhexyl salicylate, from 0.1% to 3% of ethylhexyltriazone, from 1.5% to 6% of terephthalylidenedicamphorsulfonic acid, from 1% to 5% of octocrylene, from 0.1% to 1% of drometrizole trisiloxane, from 0.1% to 3% of bis-ethylhexyloxyphenol methoxyphenyl triazine and from 1% to 3% of TiO₂ relative to the total weight of the composition;
from 5% to 10% of ethylhexyl methoxycinnamate, from 10% to 15% of terephthalylidenedicamphorsulfonic acid, from 0.5% to 4% of drometrizole trisiloxane and from 0.1% to 3% of TiO₂ relative to the total weight of the composition;
from 2% to 6% of butyl methoxydibenzoylmethane, from 3% to 7% of ethylhexyl salicylate, from 0.1% to 3% of ethylhexyltriazone, from 1% to 5% of terephthalylidenedicamphorsulfonic acid, from 1% to 5% of octocrylene, from 0.1% to 2% of drometrizole trisiloxane, from 1% to 5% of bis-ethylhexyloxyphenol methoxyphenyl triazine and from 3% to 7% of TiO₂ relative to the total weight of the composition;
from 0.1% to 2% of drometrizole trisiloxane, from 4% to 8% of homosalate, from 1% to 5% of bis-ethylhexyloxyphenol methoxyphenyl triazine, from 2% to 6% of butyl methoxydibenzoylmethane, from 1% to 5% of terephthalylidenedicamphorsulfonic acid, from 3% to 7% of ethylhexyl salicylate, and from 1% to 5% of ethylhexyltriazone.

Additives

The composition according to the invention may also comprise additives such as, for example, inorganic screening agents, active agents, fillers, fatty substances, polymers or silicones.

Inorganic Screening Agents

The inorganic screening agents are chosen from coated or uncoated metal oxide pigments in which the mean size of the primary particles is preferentially between 5 nm and 100 nm (preferably between 10 nm and 50 nm), for instance titanium oxide (amorphous or crystallized in rutile and/or anatase form), iron oxide, zinc oxide, zirconium oxide or cerium oxide pigments, which are all UV-photoprotective agents that are well known per se.

The pigments may be coated or uncoated.

The coated pigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal alkoxides (titanium or aluminum alkoxides), polyethylene, silicones, proteins (collagen, elastin), alkanolamines, silicon oxides, metal oxides or sodium hexametaphosphate.

As is known, silicones are organosilicon polymers or oligomers of linear or cyclic, branched or crosslinked structure, of variable molecular weight, obtained by polymerization and/or polycondensation of suitably functionalized silanes, and are essentially constituted of a repetition of main units in which the silicon atoms are linked together via oxygen atoms (siloxane bond), optionally substituted hydrocarbon radicals being directly attached to said silicon atoms via a carbon atom.

The term “silicones” also encompasses the silanes required for their preparation, in particular alkylsilanes.

The silicones used for coating the pigments that are suitable for the present invention are preferably chosen from the group containing alkyl silanes, polydialkylsiloxanes and polyalkylhydrogenosiloxanes. Even more preferentially, the silicones are chosen from the group containing octyltrimethylsilane, polydimethylsiloxanes and polymethylhydrogenosiloxanes.

Of course, before being treated with silicones, the metal oxide pigments may have been treated with other surface agents, in particular with cerium oxide, alumina, silica, aluminum compounds or silicon compounds, or mixtures thereof.

The coated pigments are more particularly titanium oxides that have been coated:
with silica, such as the product Sunveil from the company Ikeda and the product Eusolex T-AVO from the company Merck,
with silica and iron oxide, such as the product Sunveil F from the company Ikeda,
with silica and alumina, such as the products Microtitanium Dioxide MT 500 SA and Microtitanium Dioxide MT 100 SA from the company Tayca, Tioveil from the company Tioxide and Mirasun TiW 60 from the company Rhodia,
with alumina, such as the products Tipaque TTO-55 (B) and Tipaque TTO-55 (A) from the company Ishihara and UVT 14/4 from the company Kemira,
with alumina and aluminum stearate, such as the product Microtitanium Dioxide MT 100 TV, MT 100 TX, MT 100 Z and MT-01 from the company Tayca, and the products Solaveil CT-10 W, Solaveil CT 100 and Solaveil CT 200 from the company Uniqema,
with silica, alumina and alginic acid, such as the product MT-100 AQ from the company Tayca,
with alumina and aluminum laurate, such as the product Microtitanium Dioxide MT 100 S from the company Tayca,
with iron oxide and iron stearate, such as the product Microtitanium Dioxide MT 100 F from the company Tayca,
with zinc oxide and zinc stearate, such as the product BR351 from the company Tayca,
with silica and alumina and treated with a silicone, such as the products Microtitanium Dioxide MT 600 SAS, Microtitanium Dioxide MT 500 SAS or Microtitanium Dioxide MT 100 SAS from the company Tayca,
with silica, alumina and aluminum stearate and treated with a silicone, such as the product STT-30-DS from the company Titan Kogyo,
with silica and treated with a silicone, such as the product UV-Titan X 195 from the company Kemira, or the product SMT-100 WRS from the company Tayca,
with alumina and treated with a silicone, such as the products Tipaque TTO-55 (S) from the company Ishihara or UV Titan M 262 from the company Kemira,
with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca.

Other titanium oxide pigments treated with a silicone are preferably TiO₂ treated with octyltrimethylsilane and for which the mean size of the elementary particles is between 25 and 40 nm, such as the product sold under the trade name T 805 by the company Degussa Silices, TiO2 treated with a polydimethylsiloxane and for which the mean size of the elementary particles is 21 nm, such as the product sold under the trade name 70250 Cardre UF TiO2SI3 by the company Cardre, anatase/rutile TiO2 treated with a polydimethylhydrogenosiloxane and for which the mean size of the elementary particles is 25 nm, such as the product sold under the trade name Micro Titanium Dioxide USP Grade Hydrophobic by the company Color Techniques.

The uncoated titanium oxide pigments are sold, for example, by the company Tayca under the trade names Microtitanium Dioxide MT 500 B or Microtitanium Dioxide MT 600 B, by the company Degussa under the name P 25, by the company Wackher under the name Transparent titanium oxide PW, by the company Miyoshi Kasei under the name UFTR, by the company Tomen under the name ITS and by the company Tioxide under the name Tioveil AQ.

The uncoated zinc oxide pigments are, for example
those sold under the name Z-Cote by the company Sunsmart;
those sold under the name Nanox by the company Elementis;
those sold under the name Nanogard WCD 2025 by the company Nanophase Technologies.

The coated zinc oxide pigments are, for example
those sold under the name Z-Cote HP1 by the company Sunsmart (dimethicone-coated ZnO);
those sold under the name Zinc Oxide CS-5 by the company Toshibi (ZnO coated with polymethylhydrogenosiloxane);
those sold under the name Nanogard Zinc Oxide FN by the company Nanophase Technologies (as a 40% dispersion in Finsolv TN, C12-C15 alkyl benzoate);
those sold under the name Daitopersion ZN-30 and Daitopersion Zn-50 by the company Daito (dispersions in cyclopolymethylsiloxane/oxyethylenated polydimethylsiloxane, containing 30% or 50% of nano zinc oxides coated with silica and polymethylhydro genosiloxane);
those sold under the name NFD Ultrafine ZnO by the company Daikin (ZnO coated with perfluoroalkyl phosphate and copolymer based on perfluoroalkylethyl as a dispersion in cyclopentasiloxane);
those sold under the name SPD-Z1 by the company Shin-Etsu (ZnO coated with silicone-grafted acrylic polymer, dispersed in cyclodimethylsiloxane);
those sold under the name Escalol Z100 by the company ISP (alumina-treated ZnO dispersed in an ethylhexyl methoxycinnamate/copolymer PVP-hexadecene/methicone mixture);
those sold under the name Fuji ZnO-SMS-10 by the company Fuji Pigment (ZnO coated with silica and polymethylsilsesquioxane);
those sold under the name Nanox Gel TN by the company Elementis (ZnO dispersed at a concentration of 55% in C₁₂-C₁₅ alkyl benzoate with hydroxystearic acid polycondensate).

The uncoated cerium oxide pigments are sold, for example, under the name Colloidal Cerium Oxide by the company Rhone-Poulenc.

The uncoated iron oxide pigments are sold, for example, by the company Arnaud under the names Nanogard WCD 2002 (FE 45B), Nanogard Iron FE 45 BL AQ, Nanogard FE 45R AQ and Nanogard WCD 2006 (FE 45R) or by the company Mitsubishi under the name TY-220.

The coated iron oxide pigments are sold, for example, by the company Arnaud under the names Nanogard WCD 2008 (FE 45B FN), Nanogard WCD 2009 (FE 45B 556), Nanogard FE 45 BL 345 and Nanogard FE 45 BL or by the company BASF under the name Transparent Iron Oxide.

Mention may also be made of mixtures of metal oxides, in particular of titanium dioxide and of cerium dioxide, including the equal-weight mixture of titanium dioxide and cerium dioxide coated with silica, sold by the company Ikeda under the name Sunveil A, and also the mixture of titanium dioxide and zinc dioxide coated with alumina, silica and silicone, such as the product M 261 sold by the company Kemira, or coated with alumina, silica and glycerol, such as the product M 211 sold by the company Kemira.

Composite Particles

The composition according to the invention may also comprise, by way of inorganic screening agents, composite particles.

The composite particles that can be used according to the present invention comprise a matrix and an inorganic UV-screening agent. The matrix comprises one or more organic and/or inorganic materials. The inorganic UV screening agent is generally chosen from metal oxides.

According to one preferred variant, the matrix is essentially constituted of the organic and/or inorganic material.

The inorganic materials that can be used in the matrix according to the present invention are chosen from the group formed by mica, synthetic mica, talc, sericite, boron nitride, glass, calcium carbonate, barium sulfate, hydroxyapatite, silica, silicate, magnesium sulfate, magnesium carbonate, magnesium trisilicate, aluminum oxide, aluminum silicate, calcium silicate, calcium phosphate, magnesium oxide, bismuth oxychloride, kaolin, hydrotalcite, mineral clays, synthetic clays and mixtures thereof.

The organic materials which can be used to form the matrix are chosen from the group formed by poly(meth)acrylates, polyamides, silicones, polyurethanes, polyethylenes, polypropylenes, polystyrenes, polyhydroxyalkanoates, polycaprolactams, poly(butylene succinate)s, polysaccharides, polypeptides, polyvinyl alcohols, polyvinyl resins, fluoropolymers, waxes, polyesters, polyethers and their mixtures.

The inorganic UV screening agents that can be used in the composite particle are chosen from metal oxides.

Preferably, these metal oxides are chosen from titanium dioxide TiO₂, zinc oxide ZnO and iron oxide FeO. Particularly preferably, the inorganic UV screening agent is TiO₂.

These metal oxides may be in the form of particles with a mean size generally of less than 0.2 μm. Advantageously, the TiO₂ particles used have a mean size of less than or equal to 0.1 μm.

These metal oxides may also be in the form of layers, preferably multilayers having a mean thickness generally of less than 0.2 μm.

Preferably, the content of composite particles of the composition according to the invention ranges from 1% to 70%, preferably from 1.5% to 45%, preferably from 2% to 20% by weight relative to the total weight of the cosmetic composition.

The composite particles that can be used according to the invention are preferably spherical.

They may be hollow, smooth, rough or porous.

When the composite particles are in spherical form, they are characterized by a mean diameter between 0.1 μm and 30 μm, preferably between 0.2 μm and 20 μm and more preferably between 0.3 μm and 10 μm, advantageously between 0.5 μm and 10 μm.

The term “spherical” is intended to mean that the particle has a sphericity index, i.e. the ratio between its largest diameter and its smallest diameter, of less than 1.2.

Forms of the Composite Particles

According to a first variant, the composite particles comprise a matrix comprising an organic and/or inorganic material, in which matrix particles of inorganic UV screening agent are included. Preferably, the composite particles are constituted of a matrix comprising an organic and/or inorganic material, in which matrix particles of inorganic UV screening agent are included.

According to this embodiment, the matrix exhibits inclusions and particles of inorganic UV screening agent are placed in the inclusions of the matrix.

Preferably, the composite particles are spherical and have inclusions in which particles of inorganic UV screening agent are placed.

As composite particles corresponding to this variant, mention may be made of the products Sunsil TIN 50 and Sunsil TIN 40 sold by the company Sunjin Chemical. These spherical composite particles having a mean size between 2 and 7 μm are formed of TiO₂ encapsulated in a silica matrix.

According to a second variant, the composite particles comprise a matrix made of an organic and/or inorganic material, which matrix is covered with at least one layer of inorganic UV screening agent which can be connected to the matrix using a binder.

According to this second variant, the matrix particles are, preferably, of spherical shape.

According to this second variant, the mean thickness of the layer of inorganic UV screening agent is generally approximately ten nanometers. The mean thickness of the layer of inorganic UV-screening agent is advantageously between 10⁻³ and 0.2 μm, preferably between 10⁻² and 0.2 μm.

According to a third variant, the composite particles contain an inorganic UV screening agent covered with at least one layer of an organic and/or inorganic material. According to this third variant, the particles of inorganic UV screening agent are characterized by an average elementary size generally of between 10⁻³ and 0.2 μm. Advantageously, the metal oxide particles used have a mean elementary size between 10⁻² and 0.1 μm.

The matrix may be formed from one or more organic or inorganic materials. There may then be a continuous phase of materials, such as an alloy, that is to say, a continuous phase in which the materials can no longer be separated, or a non-continuous phase of materials, for example composed of an organic or inorganic material covered with a layer of another different organic or inorganic material.

According to one variant, in particular when the composite particles comprise a matrix covered with a layer of UV screening agent, the composite particles may furthermore be covered with an additional coating, in particular chosen from biodegradable or biocompatible materials, lipid materials, for instance surfactants or emulsifiers, polymers, and oxides.

The composite particles used according to the invention have a size of between 0.1 and 30 μm, preferably between 0.5 and 20 μm and more preferably between 0.3 and 10 μm, advantageously between 0.5 and 10 μm.

Preferably, the UV screening agent used in the composite particle is TiO₂ or a mixture of TiO₂ and ZnO.

Preferably, the matrix of the spherical composite particle contains a material or a mixture of materials chosen from:
SiO₂,
polymethyl methacrylate,
copolymers of styrene and of a C₁/C₅ alkyl (meth)acrylate derivative,
polyamides, such as nylons.

As composite particles corresponding to this variant, mention may be made of the products Sunsil TIN 50 and Sunsil TIN 40 sold by the company Sunjin Chemical. These spherical composite particles having a mean size between 2 and 7 μm are formed of TiO₂ encapsulated in a silica matrix.

Mention may also be made of the following particles:
spherical composite particles having a mean size between 4 and 8 μm, containing TiO₂ and SiO₂ and having the trade name Eospoly TR sold by the company Creations Couleurs,
composite particles containing TiO₂ and a styrene/alkyl acrylate copolymer matrix sold under the name Eospoly UV TR22 HB 50 by the company Creations Couleurs,
composite particles containing TiO₂ and ZnO and a PMMA matrix and having the trade name Sun PMMA-T50 sold by the company Sunjin Chemical.

Among the composite particles that can be used according to the invention, mention may also be made of spherical composite particles containing TiO₂ and SiO₂ and having the trade name STM ACS-0050510, supplied by the company JGC Catalysts and Chemical.

Preferably, the inorganic screening agents are chosen from titanium dioxide, optionally coated with aluminum hydroxide and with stearic acid, titanium dioxide optionally coated with stearic acid and with alumina, and mixtures thereof.

According to one particular embodiment of the invention, the at least one inorganic screening agent is present in the composition according to the invention in a content ranging from 0.01% to 20% by weight, preferably from 0.1% to 10% by weight, preferably from 0.5% to 7% by weight, and in particular from 0.8% to 6% by weight, relative to the total weight of the composition.

The compositions in accordance with the present invention may also comprise standard cosmetic adjuvants chosen in particular from organic solvents, ionic or nonionic, hydrophilic or lipophilic thickeners, softeners, humectants, opacifiers, stabilizers, emollients, silicones, antifoams, fragrances, preserving agents, anionic, cationic, nonionic, zwitterionic or amphoteric surfactants, fillers, polymers, propellants, basifying or acidifying agents or any other ingredient commonly used in the cosmetics and/or dermatological field.

Among the organic solvents that may be mentioned are lower alcohols and polyols. The latter may be chosen from glycols such as propylene glycol, butylene glycol, dipropylene glycol, glycerol, 1,3-propanediol, pentylene glycol or hexylene glycol.

Hydrophilic thickeners that may be mentioned include carboxyvinyl polymers, such as Carbopols (Carbomers) and the Pemulens (acrylate/C10-C30 alkyl acrylate copolymer); polyacrylamides, for instance the crosslinked copolymers sold under the names Sepigel 305 (CTFA name: polyacrylamide/C13-C14 isoparaffin/Laureth 7) or Simulgel 600 (CTFA name: acrylamide/sodium acryloyldimethyltaurate copolymer/isohexadecane/polysorbate 80) by the company SEPPIC; 2-acrylamido-2-methylpropanesulfonic acid polymers and copolymers, optionally crosslinked and/or neutralized, for instance poly(2-acrylamido-2-methylpropanesulfonic acid) sold by the company Hoechst under the trade name Hostacerin AMPS (CTFA name: ammonium polyacryloyldimethyl taurate or Simulgel 800 sold by the company SEPPIC (CTFA name: sodium polyacryolyldimethyltaurate/polysorbate 80/sorbitan oleate); copolymers of 2-acrylamido-2-methylpropanesulfonic acid and of hydroxyethyl acrylate, for instance Simulgel NS and Sepinov EMT 10 sold by the company SEPPIC; cellulose derivatives such as hydroxyethylcellulose; polysaccharides and in particular gums such as xanthan gum; and mixtures thereof.

As lipophilic thickeners, mention may be made of synthetic polymers such as the poly(C₁₀-C₃₀ alkyl acrylate)s sold under the name Intelimer IPA 13-1 and Intelimer IPA 13-6 by the company Landec, or else modified clays such as hectorite and derivatives thereof, for instance the products sold under the name Bentone.

Needless to say, those skilled in the art will take care to select the optional additional compound(s) mentioned above and/or the amounts thereof such that the advantageous properties intrinsically associated with the compositions in accordance with the invention are not, or are not substantially, adversely affected by the envisioned addition(s).

The compositions according to the invention may be prepared according to the techniques that are well known to those skilled in the art. They may in particular be in the form of a direct, inverse or multiple (O/W, W/O, O/W/O or W/O/W) emulsion. They may optionally be packaged as an aerosol and may be in the form of a spray.

The compositions according to the invention are preferably in the form of an oil-in-water or water-in-oil emulsion, more particularly oil-in-water emulsions.

The emulsions generally contain at least one emulsifier chosen from amphoteric, anionic, cationic or nonionic emulsifiers, used alone or as a mixture. The emulsifiers are appropriately chosen according to the emulsion to be obtained (W/O or O/W). The emulsions may also contain stabilizers of other types, for instance fillers, or gelling or thickening polymers.

As emulsifying surfactants that may be used for the preparation of the W/O emulsions, examples that may be mentioned include sorbitan, glycerol or sugar alkyl esters or ethers; silicone surfactants, for instance dimethicone copolyols, such as the mixture of dimethicone and PEG/polypropylene glycol (PPG) 18/18 dimethicone, sold under the name X-22-6711D by the company Shin Etsu, and alkyl dimethicone copolyols such as laurylmethicone copolyol sold under the name Dow Corning 5200 Formulation Aid by the company Dow Corning; cetyldimethicone copolyol, such as the product sold under the name Abil EM 90R by the company Goldschmidt, and the mixture of cetyldimethicone copolyol, of polyglyceryl isostearate (4 mol) and of hexyl laurate, sold under the name Abil WE 09 by the company Goldschmidt. It is also possible to add thereto one or more co-emulsifiers, which, advantageously, can be chosen from the group comprising polyol alkyl esters or emulsifying silicone elastomers, such as the mixture of dimethicone and cross-linked polymer of dimethicone/PEG-10/15 sold under the name KSG-210.

Polyol alkyl esters that may in particular be mentioned include polyethylene glycol esters, for instance PEG-30 dipolyhydroxystearate, such as the product sold under the name Arlacel P135 by the company ICI.

Glycerol and/or sorbitan esters that may be mentioned include, for example, polyglyceryl isostearate, such as the product sold under the name Isolan GI 34 by the company Goldschmidt; sorbitan isostearate, such as the product sold under the name Arlacel 987 by the company IC; sorbitan glyceryl isostearate, such as the product sold under the name Arlacel 986 by the company ICI, and mixtures thereof.

For the O/W emulsions, examples of emulsifiers that may be mentioned include nonionic emulsifiers such as oxyalkylenated (more particularly polyoxyethylenated) esters of fatty acids and of glycerol; oxyalkylenated esters of fatty acids and of sorbitan; oxyalkylenated (oxyethylenated and/or oxypropylenated) esters of fatty acids, such as the PEG-100 stearate/glyceryl stearate mixture sold, for example, by the company ICI under the name Arlacel 165; oxyalkylenated (oxyethylenated and/or oxypropylenated) ethers of fatty alcohols; esters of sugars, such as sucrose stearate; or ethers of fatty alcohol and of sugar, in particular alkyl polyglucosides (APGs), such as decyl glucoside and lauryl glucoside, sold, for example, by the company Henkel under the respective names Plantaren 2000 and Plantaren 1200, cetostearyl glucoside, optionally as a mixture with cetostearyl alcohol, sold, for example, under the name Montanov 68 by the company SEPPIC, under the name Tegocare CG90 by the company Goldschmidt and under the name Emulgade KE3302 by the company Henkel, and also arachidyl glucoside, for example in the form of the mixture of arachidyl alcohol, behenyl alcohol and arachidyl glucoside, sold under the name Montanov 202 by the company SEPPIC. According to one particular embodiment of the invention, the mixture of the alkyl polyglucoside as defined above with the corresponding fatty alcohol can be in the form of a self-emulsifying composition, for example as described in document WO-A-92/06778.

The examples that follow illustrate the invention without limiting the scope thereof

In the description and the examples, the percentages are percentages by weight. The ingredients are mixed in the order and under the conditions that are easily determined by those skilled in the art.

### EXAMPLES

### Example 1

### Compositions in Accordance with the Invention

The Compositions are Prepared by Mixing the Following Ingredients:

| | Composi- | Composi- | Composi- | Composi- |
|---|---|---|---|---|
| | tion 1 (% | tion 2 (% | tion 3 (% | tion 4 (% |
| INCI Name | by weight) | by weight) | by weight) | by weight) |
| Disodium EDTA | 0.1 | 0.1 | | 0.1 |
| Tocopherol | 0.5 | 0.5 | | 0.5 |
| Triethanoalmine | 0.33 | 0.85 | 0.07 | 0.59 |
| Preserving agent | 1 | 1 | 1 | 1 |
| Octyldodecanol | 2 | | 2 | 2 |
| Cetearyl alcohol | 1 | 1 | 1 | 1 |
| Diisopropyl | | 3 | | 5 |
| sebacate | | | | |
| Resveratrol | 0.25 | 0.25 | 0.25 | 0.25 |
| Ethylhexyl | | | 7.5 | |
| methoxycinnamate | | | | |
| Butyl | 2.5 | 3 | | 3.5 |
| methoxydibenzoyl | | | | |
| methane | | | | |
| Ethylhexyl | | 5 | | 5 |
| salicilate | | | | |
| Ethylhexyl triazone | 1 | 1 | | 1 |
| Terephthalylidene- | 1.5 | 4.5 | 12 | 3 |
| dicamphorsulfonic | | | | |
| acid | | | | |
| Octocrylene | 7 | 2.5 | | 2.5 |
| Drometrizole | 1.5 | 0.5 | 1.5 | 0.5 |
| trisiloxane | | | | |
| Bis- | | 1 | | 3 |
| ethylhexyloxyphenol | | | | |
| methoxyphenyl | | | | |
| triazine | | | | |
| Titanium dioxide | 1 | 2 | 1 | 5 |
| (and) aluminum | | | | |
| hydroxide (and) | | | | |
| stearic acid | | | | |
| Xanthan gum | 0.2 | 0.2 | 0.2 | 0.2 |
| Ammonium | 1.7 | 1.7 | 1.7 | 1.7 |
| polyacryloyldimethyl | | | | |
| taurate | | | | |
| Cyclohexasiloxane | 1 | | | 1 |
| Dimethicone (and) | 3 | 3 | 3 | 3 |
| dimethiconol | | | | |
| Alcohol denat. | 5 | 5 | 5 | 5 |
| Glycerol | 4 | 4 | 4 | 4 |
| Propylene glycol | 8 | 8 | 8 | 8 |
| Stearic acid | 2 | 2 | 2 | 2 |
| Glyceryl stearate | 2 | 2 | 2 | 2 |
| (and) PEG-100 | | | | |
| stearate | | | | |
| PEG-20 stearate | 0.8 | 0.8 | 0.8 | 0.8 |
| Water | qs 100 | qs 100 | qs 100 | qs 100 |

The emulsions of compositions 1 to 4 are fine, homogeneous, glossy, smooth and stable over time.

### Example 2

### Compositions not in Accordance with the Invention

| | Composi- | Composi- | Composi- | Composi- |
|---|---|---|---|---|
| | tion 5 (% | tion 6 (% | tion 7 (% | tion 8 (% |
| INCI Name | by weight) | by weight) | by weight) | by weight) |
| Disodium EDTA | 0.1 | 0.1 | 0.1 | 0.1 |
| Tocopherol | 0.5 | 0.5 | 0.5 | 0.5 |
| Triethanoalmine | 0.07 | 1.58 | 0.07 | 0.07 |
| Citric Acid | | 0.528 | | |
| Silica | 0.5 | 0.5 | 0.5 | 0.5 |
| Preserving agent | 1 | 1 | 1 | 1 |
| Octyldodecanol | 2 | 2 | 2 | 2 |
| Cetearyl alcohol | 1 | 1 | 1 | 1 |
| Isopropyl lauroyl | 7.5 | | | |
| sarcosinate | | | | |
| Resveratrol | 0.25 | 0.25 | 0.25 | 0.25 |
| Phenylbenzimidazole | | 1.7 | | |
| sulfonic acid | | | | |
| Ethylhexyl | | | 5 | |
| salicilate | | | | |
| Terephthalylidene- | | 0.15 | | |
| dicamphorsulfonic | | | | |
| acid | | | | |
| Octocrylene | | | 7 | |
| Bis- | | | | 1 |
| ethylhexyloxyphenol | | | | |
| methoxyphenyl | | | | |
| triazine | | | | |
| Butyl | | | 3 | |
| methoxydibenzoyl | | | | |
| methane | | | | |
| Ethylhexyl | | 7.5 | | |
| methoxycinnamate | | | | |
| Titanium dioxide | | 1 | | |
| (and) stearic acid | | | | |
| (and) alumina | | | | |
| Xanthan gum | 0.2 | 0.2 | 0.2 | 0.2 |
| Ammonium | 1.7 | 1.7 | 1.7 | 1.7 |
| polyacryloyldimethyl | | | | |
| taurate | | | | |
| Cyclohexasiloxane | 1 | 5 | 1 | 5 |
| Dimethicone (and) | 3 | 3 | 3 | 3 |
| dimethiconol | | | | |
| Alcohol denat. | 5 | 5 | 5 | 5 |
| Glycerol | 4 | 4 | 4 | 4 |
| Stearic acid | 2 | 2 | 2 | 2 |
| Glyceryl stearate | | | | |
| (and) PEG-100 | 2 | 2 | 2 | 2 |
| stearate | | | | |
| PEG-20 stearate | 0.8 | 0.8 | 0.8 | 0.8 |
| Water | qs 100 | qs 100 | qs 100 | qs 100 |

### Example 3

### Characteristics of Compositions 1 to 8

| | Compositions in accordance | | | | Compositions not in accordance | | | |
|---|---|---|---|---|---|---|---|---|
| | with the invention | | | | with the invention | | | |
| | 1 | 2 | 3 | 4 | 5 | 6 | 7 | 8 |
| Amount of UVA- | 4 | 7.5 | 12 | 6.5 | 0 | 0.15 | 3 | 3 |
| screening agent | | | | | | | | |
| (% by weight) | | | | | | | | |
| Amount of UVB- | 9.5 | 9 | 9 | 9 | 0 | 9.2 | 12 | 10 |
| screening agent | | | | | | | | |
| (% by weight) | | | | | | | | |
| SPF | 20 | 30 | 50 | 50 | / | 15 | 25 | 20 |
| PPD | 8 | 20 | 20 | 30 | / | 1 | 1 | 1 |
| SPF/PPD ratio | 2.5 | 1.5 | 2.5 | 1.67 | / | 15 | 25 | 20 |
| residual % of | 86 | 86 | 89 | 90 | <5 | 31 | 47 | 35 |
| trans-resveratrol⁽¹⁾ | | | | | | | | |

⁽¹⁾The residual percentage of resveratrol is measured after exposure of the composition to 5 J/cm² of UV, which corresponds to a daily dose.

The four compositions in accordance with the invention (compositions 1 to 4) have a very high residual percentage of resveratrol after exposure of the composition to 5 J/cm² of UV, which means that said resveratrol degrades very little on contact with daylight.

When the composition does not comprise any UV-screening agent (composition 5), the degradation of the resveratrol after exposure of the composition to 5 J/cm² of UV is virtually complete, which means that said resveratrol degrades a great deal on contact with daylight.

When the composition has a low UVA sun protection factor (PPD) (compositions 6, 7 and 8), a considerable degradation of the trans-resveratrol is also observed.

### Example 4

### Composition in Accordance with the Invention Free of TiO₂

| INCI Name | % by weight |
|---|---|
| Dimyristyl tartrate (and) cetearyl alcohol | 2.00 |
| (and) C₁₂₋₁₅ pareth-7 (and) PPG-25-laureth-25 | |
| Caprylic/capric trigyceride | 2.00 |
| Drometrizole trisiloxane | 1.50 |
| Ethylhexyl triazone | 1.00 |
| Butyl methoxydibenzoyl methane | 2.50 |
| Octocrylene | 7.00 |
| Prunus armeniaca kernel oil | 1.00 |
| Glyceryl stearate (and) PEG-100 stearate | 0.60 |
| Cyclohexasiloxane | 5.00 |
| Tocopherol | 0.50 |
| Propanediol | 3.00 |
| Disodium EDTA | 0.20 |
| Phenoxyethanol | 0.50 |
| Potassium cetyl phosphate | 1.00 |
| Terephthalylidenedicamphorsulfonic acid | 1.50 |
| Triethanolamine | 0.26 |
| Water | qs 100 |
| Acrylates/C₁₀₋₃₀ alkyl acrylate crosspolymer | 0.43 |
| Triethanolamine | 0.83 |
| Polyacrylamide (and) C₁₃₋₁₄ isoparaffin (and) | 1.00 |
| laureth-7 | |
| Hydroxyethyl acrylate/sodium | 0.50 |
| acryloyldimethyl taurate copolymer | |
| Alcohol not denat. | 5.00 |
| Lactic acid | 0.53 |
| Resveratrol | 1.00 |

This composition is homogeneous and stable over time.

1-19. (canceled)

20. A cosmetic or dermatological composition comprising, in a physiologically acceptable medium, at least trans-resveratrol and/or at least one trans-resveratrol derivative, and an organic screening system comprising at least one UVA-screening agent and at least one UVB-screening agent and/or at least one mixed UVA-UVB-screening agent,
said composition having an SPF greater than or equal to 15 and a PPD greater than or equal to 5.

21. The composition as claimed in claim 20, wherein said trans-resveratrol and/or trans-resveratrol derivative(s) is (are) present in a content ranging from 0.001% to 10%, relative to the total weight of the composition.

22. The composition as claimed in claim 20 or 21, wherein the SPF/PPD ratio ranges from 1 to 3.

23. The composition as claimed in claim 20, wherein at least one UVA-screening agent is chosen from hydrophobic dibenzoylmethane derivatives, water-soluble camphor derivatives, and mixtures thereof.

24. The composition as claimed in claim 23, wherein at least one UVA-screening agent is chosen from butyl methoxydibenzoylmethane, terephthalylidenedicamphorsulfonic acid, and mixtures thereof.

25. The composition as claimed in claim 24, wherein said at least one UVA-screening agent is a water-soluble UVA-screening agent.

26. The composition as claimed in claim 20, wherein at least one UVA-screening agent is present in a content ranging from 1% to 20%, relative to the total weight of the composition.

27. The composition as claimed in claim 20, wherein said at least one UVB-screening agent is a hydrophobic UVB-screening agent.

28. The composition as claimed in claim 27, wherein at least one hydrophobic UVB-screening agent is chosen from salicylic derivatives, cinnamates, β,β′-diphenylacrylate derivatives, triazine derivatives, and mixtures thereof.

29. The composition as claimed in claim 20, wherein said at least one UVB-screening agent is present in a content ranging from 1% to 20% relative to the total weight of the composition.

30. The composition as claimed in claim 20, wherein said organic screening system comprises at least one mixed UVA-UVB-screening agent.

31. The composition as claimed in claim 20, wherein the hydrophobic mixed UVA-UVB-screening agent is chosen from phenylbenzotriazole derivatives and bis-resorcinyl triazine derivatives, preferably from drometrizole trisiloxane, bis-ethylhexyloxyphenol methoxyphenyl triazine, and mixtures thereof.

32. The composition as claimed in claim 20, wherein said organic screening system comprises at least one water-soluble UVA-screening agent and at least one hydrophobic UVB-screening agent.

33. A process for stabilizing a composition comprising trans-resveratrol and/or at least one trans-resveratrol derivative, comprising a step during which an organic screening system comprising at least one UVA-screening agent and at least one UVB-screening agent and/or at least one mixed UVA-UVB-screening agent is added to said composition in an amount such that the SPF of the composition is brought to a value greater than or equal to 15 and the PPD of the composition is brought to a value greater than or equal to 5.

34. The use of a screening system comprising at least one UVA-screening agent and at least one UVB-screening agent and/or at least one mixed UVA-UVB-screening agent, for stabilizing a composition comprising trans-resveratrol and/or at least one trans-resveratrol derivative.
